(19) United States
(12) Patent Application Publication
(10) Pub. No.: US 2022/0000392 A1
(43) Pub. Date: Jan. 6, 2022

(54) LOAD-DETECTING ORTHOSIS

(71) Applicant: GOLEX AG, Basel (CH)

(72) Inventor: Frank Werner, Ueberstorf (CH)

(21) Appl. No.: 17/299,507

(22) PCT Filed: Dec. 3, 2019
(86) PCT No.: PCT/IB2019/001419

§ 371 Date: Jun. 3, 2021

(30) Foreign Application Priority Data
Dec. 3, 2018 (DE) DE10 2018 220 853

(51) Int. Cl. A61B 5/103; A61B 5/00

(52) U.S. Cl. CPC A61B 5/1036; A61B 5/6807; A61B 2562/0247; A61B 5/0004; A61B 5/746

(57) ABSTRACT

A loading-detecting orthosis includes a sensor that generates sensor signals and that has sensor elements in pressure areas. The loading-detecting orthosis further includes sensor signal-evaluating electronics that are designed to indicate critical loading. The sensor signal-evaluating electronics are designed to ascertain characteristic values regarding pressure loading events detected at least per pressure area by way of the sensor elements, to form a sum value into which the characteristic values are incorporated by amount in weighted form such that at least three different weights are used, and to generate an alert signal when the sum value exceeds a certain amount.

### CROSS-REFERENCE TO RELATED APPLICATIONS

The present application is the national stage of International Pat. App. No. PCT/IB2019/001419 filed Dec. 3, 2019, and claims priority under 35 U.S.C. § 119 to DE 10 2018 220 853, filed in the Federal Republic of Germany on Dec. 3, 2018, the content of each of which are incorporated herein by reference in their entireties.

### FIELD OF THE INVENTION

The present invention relates to the detection of a loading.

### BACKGROUND

Complications often occur following bone fractures and operations in the leg and foot region. For instance, fractures do not heal but rather have to be re-operated on, endoprostheses do not grow in, but rather become loose, etc. Medical practice has shown that, following certain orthopedic interventions, roughly 10% of all patients require new operations merely because the patient has overloaded the critical point.

There are therefore already a large number of proposals as to how to recognize overloading and warn against it.

The present inventor is for instance aware of CH 704 972, according to which the musculoskeletal system is allowed to be only partially loaded for several weeks in the post-operative course in the case of ankles, knees and hips. It has therefore been proposed that the patient wear a special orthosis containing a measuring arrangement using which force/time curves are able to be recorded for each step. It is proposed for it to be possible to set loading limits for a training mode and a maximum loading value via switches, and when loading takes place in the correct range, positive feedback is given in the form of an LED that lights up in green, and when a set value is exceeded, a warning is given by a red LED in combination with an acoustic signal or vibration.

DE 37 14 218 A1 discloses a therapeutic protection device for protecting against overloading of the human musculoskeletal system, referred to as a so-called “sole balance,” wherein it is proposed to use only a few pressure sensors, such as two pressure sensors in the ball region and one pressure sensor in the heel region in an insole. It is explained that evaluating a behavioral profile improves healing chances for the patient, and that minimal loading appears to stimulate the healing process through mechanical stimuli. It is furthermore pointed out that a low setpoint loading range can be predefined at the start of the treatment. A loading history of for example 2 weeks should be recorded.

DE 198 10 182 C1 discloses a device for detecting overloading of the lower musculoskeletal system and/or the spinal column of a person, wherein reference is made to endoprostheses and to the fact that loading that is targeted and proportioned as early as possible is beneficial to the healing process, but overloading caused by hard impacts when taking stairs quickly or jumping should be avoided. It is mentioned that overloading is registered by the load-detecting arrangement when a predetermined load threshold value is exceeded, or the load threshold value can also be selected such that the signal is already triggered when, although the loading is not yet damaging, a further increase in the loading would be damaging. It is also mentioned that load threshold values can be set individually, for example in a manner adapted to the respective osteological, muscular and/or neurological conditions and/or the ligament conditions of the person.

DE 295 12 711 U1 discloses a measuring system for static and dynamic pressure distribution measurement on the sole of a person. A data acquisition unit is proposed, by way of which pressure sensors on the force measurement soles are queried and measured data are able to be transmitted via radio. The measurement results are intended to be displayed in the form of colored isobars or in the form of pressure profiles, as a result of which “firstly transmission via radio is possible” and “loading on the sole is able to be measured with a sufficiently high resolution.” For example, 64 pressure sensors are proposed and are intended to allow a fine resolution of 1/16 N/cm² and are intended to be sampled at at least 40 Hz for slow and normal walking movements and 50 to 100 Hz for fast movements, as occur in medical sports examinations.

A pressure sensor, for example for an item of footwear, is known from DE 11 2013 002 836, accordingly WO 2013/182 633. The intention therein is to provide pressure sensors whose pressure measurement cells have an extended dynamic range, the electrical resistance thereof decreasing more slowly, but over a wider range, as the pressure increases.

Reference is also made to DE 20 2017 000 608 U1, which already discloses a loading-detecting orthosis by way of which peak loading is warned against.

WO 2015/145 273 A1 discloses a system for assessing the loading of a lower extremity with an accelerometer in order to detect shock waves from individual footsteps. The intention is to generate and process shock wave data in order to assist the user in future loading minimization.

DE 299 19 839 U1 deals with a system for detecting stepping forces, in which a patient is supposed to keep the loading within medically recommended limits. Although the storage of a temporal profile of the recorded values of the stepping forces is mentioned as well as the obtainment of force/time diagrams, a particularly recommended signal evaluation that also has a battery-preserving effect is not able to be derived from the document. WO 01/39655A3 furthermore relates to a shoe sensor arrangement for gait analysis.

### SUMMARY OF THE INVENTION

It is desirable to specify a loading-detecting orthosis of inexpensive design that is still able to output precise warnings. It is also desirable to avoid false alarms. Furthermore, as an alternative or in addition, it is desirable to enable long-term operation of a loading-detecting orthosis without changing battery or recharging rechargeable batteries.

The problem addressed by the invention is to provide novel subject matter for industrial application.

According to a first important aspect, what is thus proposed is a loading-detecting orthosis having a sensor that generates sensor signals and that has sensor elements in pressure areas, and sensor signal-evaluating electronics therefor that are designed to indicate critical loading, wherein the sensor signal-evaluating electronics are designed to ascertain characteristic values regarding pressure loading events detected at least per pressure area by way of the sensor elements, to ascertain a sum value into which the characteristic values are incorporated by amount in weighted form such that at least three different weights are used, and to generate an alert signal when the sum value exceeds a stipulated amount.

An orthosis designed in this way has the advantage that a user is able firstly to be reliably warned when the manner of his orthosis use is harmful to health, but secondly at the same time a situation is avoided whereby a user is additionally unsettled by largely singular events and after these have occurred. Specifically, excessive pressure loading events often occur when a user for example stumbles and has to regain his balance, has to swerve unexpectedly, etc. This may be critical especially for people who are not used to orthoses. In such situations, the generation of a warning signal requiring attention is absolutely counter-productive, because in such situations it is not a matter of warning the user about the situation which he knows is critical, but rather giving him the opportunity to rectify this critical situation as well as possible and thus without any distraction.

Since it is not a single event that is evaluated, but rather sum values of the events, this is made considerably easier, this being the case in particular when, as provided for, the characteristic values of pressure loading events are weighted. This will generally lead to a critical high load that is observed continually leading to a warning signal being generated, whereas singular events that are also high, although they are registered, do not need to lead directly to warnings. Since more than three different weightings are used, despite the low outlay, an already sufficiently fine classification of the influence of a brief overloading or brief high loading is possible. In an example preferred embodiment, the sensor signal-evaluating electronics in the loading-detecting orthosis are furthermore designed to classify the characteristic values through comparison with a multiplicity of threshold values, in particular per step, and to apply the classification to the weighting such that greater weightings are obtained as the characteristic values become larger.

A “too high/uncritical” assessment is thus not simply performed, but rather the actual criticality of an instantaneous pressure loading is analyzed in more detail. The alert signal will therefore typically be a warning signal; it could however also constitute an incentive signal for repeatedly correct loading. The amount that the sum value has to exceed for the alert signal to be generated is typically settable, but could also be implemented beforehand in a fixed and unchangeable manner.

Typical gait patterns lead to pressure loading in the foot region, in the case of which the different regions of the foot are repeatedly loaded to a comparatively large extent in a certain order. These loading patterns can be used very effectively to recognize steps, in some cases even such that it is possible to recognize whether a person is running/walking horizontally, uphill or downhill and/or is climbing up or down stairs. It is additionally also possible to identify the swing phase of a leg during the movement, and it is possible to recognize times at which a person is for example standing still or sitting. It is possible and expedient to record the temporal profile of sensor signals with a temporal resolution that is high enough that such events are able to be distinguished from one another, or at least some of said events are able to be identified. It is pointed out that it is possible to evaluate sensor signal pressure curves per leg, that is to say to perform this for the left and right foot in each case completely independently of one another, but that, likewise and even preferably, a common evaluation can be performed because, when walking, one leg is typically not loaded during the swing phase, whereas the opposing foot is loaded to a greater extent at these times, specifically, in the case of an otherwise healthy user, such that a rolling movement takes place across the sole, which is why it is advantageous for sensor elements to be provided in multiple pressure areas.

Step recognition helps in particular to distinguish between repeatedly excessively high loading in standard situations, such as walking movements, and excessively high peak loading caused by singular events. This can likewise be applied in the weighting, because even a loading that possibly exceeds the expedient setpoint loading to a small extent has a greater effect when walking than individual high singular loading.

It is furthermore emphasized that, in the typical and advantageous implementation, not only the limits or thresholds of the sum values starting from which an alert signal is generated are settable, but rather that the weighting is advantageously also settable. It is thus able to be ensured that loading in particular foot areas is assessed as being particularly critical, this being particularly advantageous for certain treatments, for instance following metatarsal fractures. It is possible here to achieve a more accurate adaptation to the respective requirements of the patient through corresponding evaluation or weighting of the pressure areas.

It is furthermore pointed out that the alert signals can also be output in an easily understandable form when it is necessary to output a preliminary warning, for instance because a particular loading, although critical, is not yet highly dangerous. In such a case, a green light can for instance be displayed for as long as the sum value has not yet exceeded a first threshold; the green light can for instance be implemented as a green LED that is excited or for instance as a green surface on a display, such as the display surface of a smartwatch or a smartphone. Then, when the sum value exceeds a first threshold, a yellow light can be displayed instead, again for example by exciting a yellow LED or by displaying a yellow surface on a display. As soon as the sum value also exceeds a second threshold that is greater than the first threshold, then a red light is preferably displayed instead, again for example by an LED or a light-up surface on a display. It is pointed out that the LEDs or surfaces can be arranged spatially in the manner of a traffic light. The user is thus given the possibility of checking his movement behavior at any time through a brief glance and in particular, when it is not necessary to output any warnings, of recognizing problem-free functioning.

It is pointed out that the generation of an alert signal can be triggered not only when the sum value exceeds an amount critical to the health of the user in a disadvantageous manner, but rather that an alert signal can also possibly be generated when correct loading has been identified repeatedly and in the manner weighted according to the invention. This is initially expedient in a very large number of patients, since many healing processes are actually supported by regular but comparatively low loading. In the case of a traffic light arrangement as has been described above, a yellow light could for instance accordingly also be displayed for as long as a user is barely moving or moving only to an insufficient extent; when he performs a sufficient movement and for instance has reached a predefined number of steps in a certain time period without being subject to overloading, the display could change to green; where a preliminary warning is necessary, a yellow and red display, for example by simultaneously exciting two display fields or two LEDs, would be possible, and as soon as a highly critical loading situation with frequent excessive overloading is detected, an exclusively red display can be implemented.

An alert signal that encourages the correct behavior of a patient can also for example be generated where patients are intended to be trained with a type of gait more beneficial for them over the long term; it is mentioned for instance that patients with cerebral palsy perform a rolling movement of the foot, referred to as steppage gait, which leads to considerable problems over the long term. It is pointed out in this regard that children who run on the forefoot over a relatively long period of time are referred to as habitual tiptoe walkers. If this habit persists over a relatively long period of time, this can cause structural changes in the growing skeleton; the gait also has a stigmatizing effect with increasing age. Tiptoe walking persists in only about 20 percent of affected people over the age of 10; at the same time, it is expedient to treat affected children early, since treatments implemented earlier lead to greater treatment success. However, the treatment is difficult since children suppress the gait problem voluntarily at least in the short term and can display a gait with heel-first contact, which complicates the objectification of the pathology and prevents clear criteria for assessing or classifying the gait problem. This is also reflected in therapeutic concepts, in which there is no clear recommendation and which therefore rely at present, inter alia, on stretching exercises, gait training, inserts or orthoses, and also on an operative calf muscle extension or the injection of botulinum toxin type A.

Such patients are able to be trained with a normal walking behavior that is more beneficial to them using the orthosis according to the invention, for which purpose use can be made of the loading-detecting orthosis according to the invention, using which a loading is ascertained per pressure area and a sum value for a plurality of successively occurring pressure loading events is determined in a weighted manner. In this case, for instance, a positive feedback signal can be output as alert signal to a young patient training a healthier, normal type of gait. It is pointed out that the amount that the sum value has to exceed in order to trigger generation of an alert signal can vary according to training progress. Thus, at the beginning of training, an alert signal can be generated even after only a few, possibly immediately successive steps with correct loading, such as 3-5 steps, whereas an alert signal, following a longer period of training, is generated only when a larger number of steps, such as 10, 20, 50 or 100 steps are completed with correct loading or with only a few intervening steps causing incorrect loading during the sequence of steps. An appropriate adaptation as to when a positive alert signal that encourages the patient is output can be made by changing the stipulated amount, as performed for example by a physiotherapist. The number of steps that have to be completed without any mistakes before an alert signal is generated can in this case also be determined with regard to how precisely the individual steps correspond to a correct step, that is to say how accurately the pressure loading ascertained on the foot corresponds to that of correct walking behavior. This is easily possible as a result of the weighting. It can also be possible to change the evaluation scheme, for instance in that an already accumulated value is reset to zero whenever either an individual incorrect step with an incorrect rolling behavior or an incorrect pressure loading pattern has been detected—in this case the required number of steps to be performed in succession without any mistakes starts anew—or a positive alert signal encouraging the patient has been output.

At the same time, it is possible to detect whether the loading in the individual pressure areas corresponds only approximately or very well to a correct gait; this can be taken into consideration through the weighting in the sum value.

For a subsequent training phase, it is possible to implement a counter for incorrect loading and a counter for correct loading, both of which are compared with threshold values. In such cases, especially in the case of patients whose training is well advanced, mechanical assistance can possibly be largely or completely dispensed with; the orthosis effect of guaranteeing a correct foot position when walking accordingly results from the acoustic, optical or tactile feedback to the user given by the alert signal. In typical cases, the orthosis will however be a mechanically assistive orthosis that is designed to mechanically support an extremity, in particular a foot and/or ankle and/or lower limb, such that an anatomically correct position is retained or adopted. It is pointed out, with regard to such counters, that it can also be advantageous to detect correct steps, on the one hand, and steps that cause overloading, on the other hand, in order to ascertain whether enough positive stimuli are generated for a beneficial healing process. It is pointed out that, for instance in the case of particularly heavy, for instance obese patients, other threshold values can possibly be used.

In one particularly preferred variant, it is jointly evaluated whether very high values occur in quick succession or repeatedly but in individual short episodes that are far apart from one another in time. It can thus be the case that a person is carrying shopping home and in the process subjects himself to excessive loading, which leads to a high temporal density of very large characteristic values, or that individual steps are deliberately taken, which likewise leads to higher loading, in particular for people who are not yet proficient enough with mobility aids (crutches), wherein the overloading events occur less often in succession when climbing stairs than for instance any caused by carrying shopping.

Taking into consideration the temporal density of characteristic values above certain threshold values can for instance take place by allowing pressure loading contributions to the sum value to subside, by calculating a sliding average value or by allowing such pressure loading events to subside faster or on their own, these no longer being purely beneficial for healing but not yet on their own having to be assessed as critical—the absolutely uncritical contributions and the individual contributions that are still uncritical could for example subside, while highly critical ones do not subside. It is thus possible for example to calculate a sum value in which the individual contributions to the overall sum are weighted not only in accordance with the amount of the characteristic values, but rather the age is additionally also taken into consideration in each characteristic value. With regard to the subsidence, the contribution of a characteristic value to the sum value can thus for example decrease linearly over time, in particular to 0 for very old characteristic values, or to a fixed low value, such that for instance overloading during the immediately preceding steps, for example the immediately preceding 5, 10 or 20 steps is weighted for example twice as high as the 5, 10 or 20 steps prior to the immediately preceding ones; the 5, 10 or 20 steps in turn before these can be weighted half as high again, etc., this continuing until a weighting has dropped to only one percent, 5 percent, 10 percent or the like with respect to the significance of the immediately preceding steps performed. The significance of overloading a very long way back decreases accordingly for the sum value. It is pointed out that slight overloading can subside more quickly than high overloading or that particularly high overloading does not actually need to subside at all. It is pointed out that subsidence behaviors other than those explicitly mentioned can likewise be implemented. It will become apparent from the above disclosure that the subsidence is a process implemented by digital data processing, in particular by appropriate multiplication operations acting on characteristic values, and should in particular be performed and implemented explicitly as such. It should also be borne in mind that the subsidence takes place in addition to the weighting of the characteristic values. This is especially significant where the subsidence behavior is influenced by the weighting, since pressure events of different weightings should subside at different rates.

In one particularly preferred variant, at least 4, preferably at least 5 threshold values are used for the weighting. In addition to the threshold value “uncritical” and the threshold value “extremely critical,” which should be selected for pressure loading that leads to a very considerable rise in the healing risk within a very short time, that is to say within a few steps, it is possible to distinguish between threshold values for example for only slight exceedance of the setpoint loading and for a considerable pressure loading that does not however, on its own, have any direct consequences caused by individual episodes. If for instance a loading that encourages healing is 10 kg and a known excessive loading is 30 kg, intermediate thresholds can be selected for instance at 17 kg and 25 kg; in the case of a more accurate desired resolution, the threshold values can be defined in respective 5 kg steps or in a more refined manner. The weighting is preferably performed for the minor loading exceedances at least linearly, preferably more than linearly, that is to say for instance quadratically, such that a value of 1 is added to the sum for a simple exceedance, a value of 2 is added for a slightly higher exceedance and a value of 3 or 5 is added to the sum value for a particularly large exceedance. It is however pointed out that a or the very large value does not necessarily have to contribute to a much larger sum.

The loading-detecting orthosis can easily be designed inexpensively, since no specific adaptation to different patients is generally necessary. Due to the fact that only a few pressure areas are evaluated over the whole surface or averaged over certain surfaces, a standard sensor is already sufficient for virtually all patients, at least all patients in a size group. It thus becomes possible for instance to dispense with insoles that need to be adapted just to one shoe size and to use for the left or right shoe, it even being possible to easily perform standardization to one of multiple shoe size groups.

No more than four, preferably no more than only three, in particular only two pressure areas need to be provided in a corresponding insole or the like or an orthosis using same. It will generally be sufficient for one pressure area to be arranged underneath the heel and a second pressure area at the ball. Two individual sensors or small sensor fields can be provided on the ball in order to be able to perform measurements at different locations of the ball, that is to say firstly closer to the outside of the foot and secondly closer to the inside of the foot. Where more than two pressure areas are provided, pressure areas can be provided in particular on the outside of the foot halfway between the heel and the ball and close to the arch of the foot, in particular for people with a poorly developed arch, which can be beneficial especially for patients with a predisposition, in order to recognize a critical gait pattern or the loading patterns that are particularly critical for the use of certain prostheses or endoprostheses.

It will be preferred to provide no more than four, in particular no more than three, in particular one or two sensor elements per pressure area. Reducing the number of sensor elements not only reduces structural outlay but also contributes to keeping the amount of data to be evaluated low; by virtue of the proposed weighting of the characteristic values and the formation of the sum value, this reduction in evaluated information is uncritical for detecting critical loading. The electronic evaluation required in view of the low number of sensor elements, in particular through analog-to-digital conversion, is advantageously much less complex and can thus be easily implemented using only inexpensive integrated circuits that are also energy-saving in terms of operation. The sum value formation and weighting, even when subsidence is taken into consideration, sliding average values are formed, etc., is usually highly uncritical in terms of energy because it is sufficient, even precisely where step patterns have to be recognized and a large number of digital values per step, for example between 16 and 512 values per half-step, have to be evaluated, to ascertain particular characteristic values from one step, this being possible through maximum consideration, surface integration close to or at the maximum, etc., and thereafter only very few values need to be taken into consideration, these additionally needing to be processed slowly, specifically at most roughly at the speed of the step sequence. The required clock rate of a data processing arrangement is thus particularly low, which, as is known, reduces energy consumption.

It is not only possible but even favorable, if not possibly also mandatory, to arrange the pressure sensor or the individual sensor elements in an insole that equalizes loading or damps pressure peaks. This has the advantage that, in terms of data evaluation, the outlay for averaging is able to be reduced by virtue of the fact that fewer values need to be taken into consideration and that, in addition, the dynamics to be taken into consideration in the analog-to-digital conversion need to be smaller and differences from user to user turn out smaller. Equalization by the insoles distributes pressure spatially and also, because it damps loading, temporally.

Although it is possible to install the sensors in standard insoles, it is also possible to use dedicated insoles for respective patients, for instance for compensating flat feet, bent and fallen foot positions, etc. Since the insole equalizes the pressure peaks and at least partially spatially distributes standing forces, the precise position of the sensors is comparatively uncritical and can be calibrated if necessary particularly easily with very little effort, such that use in specialist orthopedic companies is easily possible. Elastic cushioning that equalizes pressure loading is thus typically present in the sole.

It is preferable for the evaluation electronics to have two regions, specifically firstly a circuit close to the sensors and in which the pressure signals from the sensor elements are conditioned, digitized and possibly partially evaluated, for instance through (weighted) sum formation, and then fed to a separate receiver via an in particular wireless interface. The division such that a significant preliminary evaluation takes place locally close to the foot has the advantage that the high-energy data transmission is required only infrequently and for small amounts of data. It is in particular not necessary to transmit data regularly as long as they are uncritical. Rather, buffer storage close to the sole is easily possible, since the evaluated or pre-evaluated data require only little memory.

It is also possible to supply power to electronic parts close to the sensors through energy harvesting. This is promoted by the fact that the data rates are low, the number of sensors is low and, moreover, data need to be transmitted only relatively infrequently. It is thus possible to provide means that are known per se to perform energy harvesting, for example from the movement of the user.

It is furthermore possible to perform calibration in order to use the characteristic values, obtained per pressure area, to conclude as to actually present real load. Such calibration can be performed statically because standing loading is easily coupled to the dynamic loading occurring when walking. A permissible setpoint value can possibly also be predefined together with a calibration, or in the case of healing processes, a setpoint value profile of permissible maximum loading or characteristic value thresholds can be predefined. It is understandable that such setpoint value profiles can however also be set independently of calibration.

It is pointed out that, in addition to the loading-detecting orthoses, insoles for these or for footwear to be worn for example post-operatively as such are also protected; in this case, the insole will then comprise the sensor elements and at least some of the sensor signal-evaluating electronics and/or connections for forwarding sensor signals or conditioned sensor signals. An insole part, for example a layer to be installed in an insole and containing the sensor elements and at least some of the sensor signal-evaluating electronics and a line or connection for forwarding sensor signals or conditioned sensor signals can furthermore be provided for example for orthopedic shoemakers.

The invention is described below purely by way of example with reference to the drawings.

### BRIEF DESCRIPTION OF THE DRAWINGS

FIG. 1 shows a loading-detecting orthosis according to an example embodiment of the present invention.

FIG. 2 shows a horizontal section through a sensor insole according to an example embodiment of the present invention.

FIG. 3 shows evaluation electronics for the sensor of FIG. 2 according to an example embodiment of the present invention.

FIG. 4 shows a unit for generating warning signals according to an example embodiment of the present invention.

FIGS. 5a-c show an illustration of the calibration of a sole sensor for a loading-detecting orthosis according to FIG. 1, according to an example embodiment of the present invention.

### DETAILED DESCRIPTION

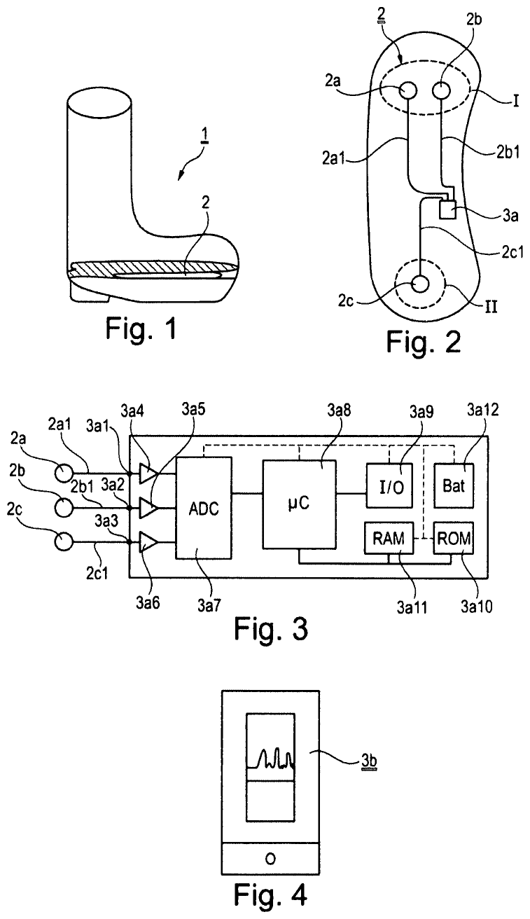

According to FIG. 1, a loading-detecting orthosis 1, referenced generally by 1, comprises a sensor 2 that generates sensor signals and that has sensor elements 2a, 2b, 2c (cf. FIG. 2) in pressure areas, indicated by dashed circles I and II, and sensor signal-evaluating electronics 3 therefor that are designed to indicate loading critical to the orthosis user, wherein the sensor signal-evaluating electronics 3a, 3b (cf. FIG. 4) are designed to ascertain characteristic values regarding pressure loading events detected per pressure area by way of the sensor elements 2a, 2b, 2c, to form a sum value into which the characteristic values are incorporated by amount in weighted form such that at least 3 different weightings are used, and to generate an alert signal when the sum value exceeds a certain amount.

The loading-detecting orthosis 1 in the illustrated exemplary embodiment is in this case an orthosis able to be used for example following a tibia fracture, in which the bones are held in the correct position in relation to one another following the fracture so that they fuse back together again correctly. During the healing process, it is possible for the user to walk using the orthosis 1, but said user has to take care that he does not overload the healing fracture when walking. He will therefore typically support himself using mobility aids such as crutches or the like in order to keep the loading on the healing leg low. Depending on the progress of the healing, the maximum permissible loading will increase again over time until the user is able to subject his leg to full load again and no longer requires the loading-detecting orthosis. Until this time, on the one hand, regular but low loading in accordance with medical knowledge is expedient for encouraging the healing process; overloading should at the same time be avoided.

Such overloading can occur because the user systematically ignores the weakness of the healing point and for instance uses a mobility aid, such as a crutch, only on one side instead of on both sides as recommended by a doctor, because he is carrying heavy items or the like. It can however also be the case that he stumbles for instance during normal cautious walking and has to use the weak leg to regain balance, which can likewise lead to pressure loading that is critical.

The sensor 2 is then incorporated, with the sensor elements 2a to 2c arranged in the pressure areas I and II, in an insole for the loading-detecting orthosis. The insole can be a standard insole that is structured with a cushioning foam or polymer material, if necessary has warming layers toward the inside of the foot, and is designed to be breathable in a manner known per se in order to reduce foot perspiration and the like.

It is pointed out in particular that the sensor elements 2a to 2c and that part 3a of the evaluating electronics to be provided in the insole and the connecting lines therefor and/or the connections and/or the energy supplies take up only a small amount of space and in particular cover or take up only a small surface area of the insole. It is additionally possible to arrange the individual slightly larger-area components, specifically the sensor elements 2a, 2b, 2c and the part 3a of the evaluating electronics, at a distance from one another and to arrange only very thin and also flexible lines between them. This improves the breathability of a corresponding insole, because the impaired surface unavailable for breathability due to the components remains small. The additional use of flexible films as carriers for integrated circuits provided in the insole is also disclosed as being possible and preferred.

It is additionally possible to arrange the sensor elements underneath a covering and damping layer facing the foot, in particular, as shown in FIG. 1, directly facing a harder, that is to say less elastic sole region of the orthosis. This is already advantageous because unpleasant pressure loading for the user caused by the sensor elements, which are slightly harder in comparison with the (sole) surroundings, is avoided. The sensor thus does not lead to impairments with regard to the walking sensation.

It is also possible to use a lower sole layer in which the corresponding sensor elements and the corresponding part 3a of the evaluating electronics are provided, and to individually install an insole adapted to a foot above this lower or lowermost insole layer. The fact that, in such a situation, ultimately the sensor elements etc. however do not need to lie directly on the outer surface, but can also be covered from below in order to protect them, is also disclosed as advantageous.

Regardless of the possibility of manually creating individually tailored insoles, in particularly advantageous example embodiments, a standardized insole is used, this having to differ only in terms of the left or right foot and a shoe size or shoe size group. In addition, in particular when very large article numbers are produced overall, it is possible to draw distinctions in order to provide more sensitive or less sensitive sensor elements for more lightweight or heavier users having the same or different shoe sizes; it is pointed out that, instead of sensor elements of differing sensitivity, cover layers or underlayers that distribute pressure to differing extents can also be used.

The pressure areas I and II are selected such that the loading that occurs when the user is walking and standing on the heel or the ball of the foot is able to be detected in an optimum manner. The sensor 2c can for example be arranged where, in a large number of healthy users, the highest average loading occurs in the heel region when said users are standing or walking. The greatest loading when standing for a large number of users can be ascertained for example using conventional blueprint technology, in which a user stands on a sheet of copier paper the bottom of which is colored blue and underneath which a white sheet of paper is arranged. The greatest loading for typical users can then be evaluated through photogrammetry or the like and averaged. Evaluation using modern means is understandably also possible, if not absolutely necessary.

That part 3a of the evaluating electronics that is arranged at or in or on the insole is then designed and active as follows:

The signals received from the sensor elements 2a, 2b, 2c via lines 2a1, 2b1, 2c1 are channeled from interfaces 3a1, 3a2, 3a3 to signal conditioning stages 3a4, 3a5, 3a6, currently indicated here in the form of amplifiers, where they are amplified and filtered if necessary, for example in order to reduce noise components caused by high-frequency components, etc. Impedance matching can also be performed, this being advantageous in particular where the sensor elements 2a to 2c are formed as resistive elements whose electrical resistance changes with active pressure. Appropriate sensor elements are known, but it is pointed out that other pressure-sensitive elements can likewise be used, such as for example strain gages etc.

The conditioned sensor signals are fed to an ADC 3a7 that selectively has enough inputs on which a conversion from analog to digital can be performed in parallel; there is preferably however cycling or switching between all of the individual inputs. It is pointed out that the conditioned sensor signals do not necessarily have to be fed individually to dedicated inputs of an analog-to-digital converter, but rather that it would also likewise be possible to connect a multiplexer or the like upstream of the ADC 3a7. It is also pointed out that conventional analog-to-digital converters are easily capable of sampling at several 10 kHz even when inexpensive analog-to-digital converters are involved, this generally being more than sufficient when cycling is intended to be performed between different sensors, given the relatively slow movement specifically for physically impaired users. This applies even where brief loading peaks occur, for instance caused by strong sudden stamping or the requirement to regain balance after stumbling. ADC accuracies of 8 bits, preferably 10 or 12 bits, are easily sufficient.

The digital signals from the analog-to-digital converter 3a7 are fed to a microcontroller 3a8, whose output is in turn routed to an I/O interface 3a9. The microcontroller 3a8 is assigned a non-volatile read-only memory ROM 3a10 and a random access memory RAM 3a11. The corresponding parts are supplied with power from a power supply 3a12, as indicated by the dashed lines going from the power supply 3a12 to the individual units 3a1 to 3a11.

The battery does not need to be directly on a circuit board or a flexible film close to the other components to store energy. Depending on how long the battery is intended to supply power to the circuit, it may be advantageous to arrange a slightly larger energy storage arrangement, for example a button cell, at a slight distance, in particular in an easily exchangeable manner, and/or there can be provision to provide an energy harvesting arrangement that obtains energy from the step movements, specifically in a manner sufficient to supply power to the arrangement.

It is pointed out that the microcontroller 3a8 has conventional circuits such as for example a clock for recording a current time, such that pressure event-related data or characteristic values can be stored in a manner provided with timestamps.

The ROM 3a10 contains program modules that make it possible, when executed on the microcontroller 3a8, to identify steps in the profiles of the signals obtained from the ADC and to recognize, in steps or per time period, pressure loading events, at least per pressure area, on the sensor elements 2a, 2b for the pressure area I or 2c for the pressure area II. The ROM 3a10 can furthermore contain information in order to prompt the microcontroller 3a8 to examine the digitized characteristic values per step or per time period, in particular when stationary per time period, for peak values and to identify these.

The ADC 3a7 needs only for instance a sampling frequency of for example 100 Hz per sensor, which is easily sufficient, especially in the case of physically impaired people, to record 15-30 values per step without any problems. This makes it possible to also possibly consider multiple values in averaged form in the peak value determination, for example around an approximate peak, in order thereby to reduce noise effects, sampling-induced effects etc. 3-6 values can for example be combined, and the respective peak loading can then be detected. The microcontroller 3a8 is also able to be programmed, either before the peak value calculation and/or after the peak value calculation, to determine an average value of the two sensor elements 2a, 2b that belong to the pressure area I. This can be achieved either through joint evaluation of the sensor element signals, that is to say through the joint signal conditioning and signal conversion, unlike what is shown, or else a pressure peak is identified in each case separately for each sensor element 2a, 2b in order then to offset the pressure peaks with one another, for example through averaging. The latter has the advantage that critical or atypical loading is recognized even better. It is also pointed out that, instead of pressure peak averaging, the temporal average values of the pressure loading detected in each sensor element can also be offset, in particular averaged, across the sensor elements of a pressure area.

The microcontroller 3a8 can furthermore be designed or programmed to correct the ADC values that are obtained from the analog-to-digital converter as output signals. Specifically, it will be understandable that it is desirable to be able to offer inexpensive sensors; this can however result in the reproducibility of data decreasing. Especially because overloading is intended to be reliably avoided, it is then advantageous to detect actually occurring pressure loading in a more precise manner.

Different loading can thus occur on each of the sensor elements 2a, 2b in spite of the same stepping force on the ground, for instance depending on exact foot posture, ball surface area, etc. It is possible to compensate this by loading the loading-detecting orthosis with a defined force following insertion of the insole by the user. Such a force can be estimated well by stepping on a set of weighing scales or the like. It has proven here that, although repeated stepping with the same force leads to reproducibly identical sizes of the pressure loading events for one and the same user, differences are observed from user to user, specifically partially with regard to the overall size of the respective events and partially with regard to the distribution of the pressure loading on the various sensor elements. It is understandable that this is to do inter alia with the standing and walking habits and for example the shape of the foot, for example due to callus-induced hardening in the ball region, etc. It is accordingly sufficient to perform calibration with static forces or loading, even though overloading should be expected when walking due to the dynamic forces occurring in the process.

It is thus advantageous to first of all record measured values and to calibrate the pressure event signals from the sensor elements on the basis thereof. This makes it possible to perform more precise weighting of the characteristic values, in particular for particularly lightweight people and/or for people with atypical foot shapes.

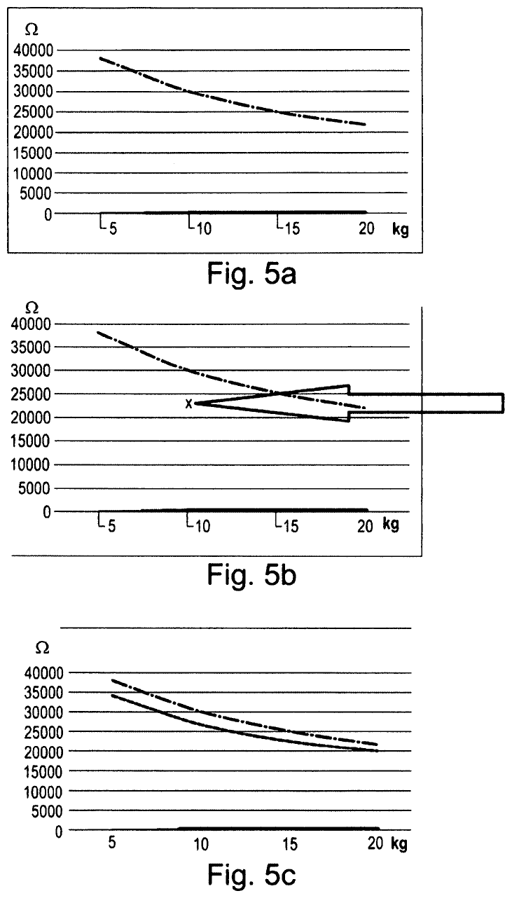

The arrangement is therefore designed to be put into a calibration mode via an operating device such as a cell phone, see FIG. 4, and the I/O interface 3a9, in which calibration mode the pressure sensor signals are stored, and then to receive an actually achieved associated loading value via the interface 3a9. For this purpose, it is possible to determine individual values for individual pressure loading and to calibrate the overall curve on the basis of these individual values, as indicated in FIG. 5. In this case, FIG. 5a illustrates a standard calibration curve that illustrates the resistance profile as a function of a loading of the sensor; FIG. 5b shows that a measured value for a particular user in the case of a measured loading of 10 kg there exhibits considerably lower resistance values than expected according to the standard curve, and FIG. 5c illustrates a correspondingly corrected calibration curve that was determined from a corresponding calibration table.

Such a calibration can be performed as follows: A loading that is uncritical for the user, and with which a healing leg can thus be loaded for as long as desired, is first of all determined. The user, using only the leg on which the orthosis is located, then steps on a sufficiently sensitive weighing scales, said user loading the corresponding leg to an increasingly great extent until the predefined loading is reached. (The loading indicated on the weighing scales will understandably consist firstly of the loading caused by the orthosis and secondly caused by the placing of the leg in the orthosis; a loading on the scales that is higher by the weight of the orthosis can accordingly possibly be selected, or a measurement of the weight of the orthosis is taken beforehand.) As soon as the desired loading is reached on the weighing scales, a calibration can be triggered. This can be performed by the user himself by operating a smartphone or the like having a suitable app, or by an assistant. It is pointed out that a weighing scales could possibly also trigger a corresponding signal.

From the calibration at a fixed weight value, a sensor value can then be used to conclude as to the actual loading. A user-specific calibration table (or, as illustrated, calibration curve) can be determined using a known general calibration curve incorporating firstly the actual sensor signal values for a known loading, and at least one user-specific calibration value.

It is pointed out that incorporating a single user-specific calibration value is generally sufficient because the loading distribution does not change significantly with increasing loads in the load range critical for healing.

The calibration, respectively a calibration table, can be stored in the RAM 3a11.

It is thus possible to correlate the electronic measured signals with at most very little outlay with loading actually occurring for a particular user. It is useful here that, even with few sensor elements, the loading pattern determined on the foot for one and the same user generally also remains the same for different loading and scales well with the overall loading.

The arrangement is furthermore designed to obtain a maximum value for the loading via the interface 3a9 and to store this in the RAM 3a11. It is pointed out that it is preferred and possible, instead of a temporally fixed value that is defined as an absolute upper limit for loading, to define a temporal upper limit profile that specifies how the maximum permissible loadability should increase over days and/or weeks. It is furthermore pointed out that either the lower threshold values can be derived from the defined upper limit for loading, for instance through a percentage-based reduction by 10%, 20%, 30% or 25% and 50% or by for example ⅕, ¼, ⅓ and ½, or else that suitable additional threshold values can be jointly stored, this having the advantage of allowing better adaptation to particular patients. More precise adaptation of the further threshold values to the maximum loading is thereby in particular made possible depending on the respective intervention, that is to say specific fracture, specific operation etc.

The microcontroller 3a8 is furthermore designed firstly to store the detected peak values of the pressure loading in the RAM 3a11, possibly temporally averaged over the pressure peak and spatially averaged over the pressure area, specifically preferably together with time information; individual values, in particular per episode, and particularly large individual peak events are also stored. The events are at the same time already assessed in the sole as to whether, individually or taken together, they indicate critical loading or overloading. This can be performed where a complete evaluation according to the invention is not intended to be performed, for instance in order to keep the computational burden close to the sole lower, for example such that only a conservative estimate is performed, for instance with equal weightings for any loading exceeding one of the thresholds or a sum value formation is performed without taking into account a subsidence behavior.

The arrangement is designed to transmit the data to a smartphone, a smartwatch or a similar mobile device via radio, in particular via Bluetooth. Connections are set up, etc., for this purpose, as is conventional. This is performed either upon request from the mobile device or actively by the insole, in particular when the event store there is already largely full or when particularly critical or a large number of almost critical events are observed.

It is also pointed out that the mobile device can on the one hand output an alert signal to the patient of the loading-detecting orthosis; it is pointed out that such a signal output does not need to be performed directly by the mobile device. It is thus possible for example for the loading-detecting orthosis itself to communicate not only with the smartphone of the user (wherein, in preferred variants, communication can in particular also be set up with devices that are used by doctors, physiotherapists, orthopedic shoemakers etc. for the initial or repeated setting of limit values and weightings), but that the smartphone itself can in turn be connected to other devices, for example an alert signal transmitter, for instance a smartwatch having a vibrating alarm that gives the user a tactile alert signal on his wrist. As an alternative or in addition, it is also possible for signals to be forwarded from the mobile device, such as the smartphone, to a control center. This does not necessarily have to take place in real time, in particular where only a statistical evaluation is intended to take place, for instance in order to obtain additional findings about general healing processes of patient cohorts or to check, over longer intervals such as daily or weekly, whether a patient is moving enough, has subjected himself to overloading and so on. In this case, a central configuration or reconfiguration can then also be performed remotely, such that the patient himself is relieved from such configuration tasks, but his aid is nevertheless adjusted with regard to a duration that has elapsed since an intervention or accident or observed diagnostic success. The data accrued in the case of large numbers of patients then also help to define loading that is typically still permissible, limit values and so on. Where at present acceptable limit values still have to be estimated by a treating doctor or physiotherapist to a more or less precautionary extent, a value that takes better consideration of experiences with other patients is able to be proposed or specified by using a database, for example a value that was uncritical for patients of a similar age or similar physical constitution and comparable injuries and did not lead to any problems, including not taking into consideration occasionally occurring exceedances of the limit values. It is pointed out that a remote configuration can be performed both by medically trained personnel such as doctors or physiotherapists, but also possibly by machine, as long as there are no approval-based obstacles to this. It will be easily understood that particular circumstances can be taken into consideration in the remote configuration as well, for instance adapting the limit loading in the case of very heavy patients.

It is also pointed out that, based on the analysis of the loading data, it is possible not only to generate warning signals, but that there is possibly also the option of recognizing the extent to which a patient requires further training. It is thus possible for instance to easily recognize events in which the patient is going up or down stairs. It is known that problems can occur particularly often when climbing stairs; training for climbing stairs is therefore performed separately in many cases. When it is established that problems occur specifically when using stairs, this being possible using the loading-detecting orthosis, the patient can for instance be requested to take specific training for climbing stairs. This is possible in principle for example by transmitting loading data to a control center and analyzing them there—preferably automatically. The result of an automatic analysis can then be indicated to an experienced professional, for instance a physiotherapist, who then contacts the patient if necessary and schedules (post-)training. This can possibly also be performed fully automatically. One alternative to transmitting data to a control center and analyzing the data in the control center is that of performing analysis locally, for instance on the smartphone of the user, and, if the analysis indicates that particular problem areas are present in a patient, for instance insufficient stair climbing, the patient can then be given the incentive or possibility, if necessary, of contacting a physiotherapist for post-training, of viewing training videos or the like. Such a local evaluation is understandably particularly advantageous where patients are particularly concerned with their data being protected.

It is furthermore pointed out that the loading-detecting orthosis according to the present invention has already collected data that indicate that problems were observed even in those patients for which, although loading classified as critical by the doctor had not been exceeded more often than for other patients, very high loading below the maximum permissible loading recommended by the doctor has still occurred on a regular basis. This demonstrates firstly that an orthosis of the present invention that detects frequent, high loading, even if this loading is not critical on its own, can contribute to significantly reducing post-treatments and at the same time be able to better specify limit loading.

The evaluation unit in the mobile radio device is then designed, upon receiving data, to form a sum value into which the characteristic values from the unit 3a are incorporated by amount in weighted form such that at least 3 different weightings are used, and to generate a warning signal when the sum value is too large. The evaluating electronics 3b in the mobile radio device (FIG. 4) are designed to compare the obtained characteristic values with threshold values and possibly to classify them. This can in particular be performed step by step.

It is possible to trigger transmission of data only whenever a first threshold, as indicated in a (preliminary) evaluation in the component 3a close to the sole, is exceeded and/or a (preliminary) evaluation on the part 3a close to the sole shows overall that critical loading is exceeded repeatedly.

An overall evaluation can also be performed close to the sole, and transmission of suitable data to the unit 3b can take place only when a for instance acoustic or vibration signal is intended to be indicated to the user. This often saves overall on energy due to the lower data transmission expenditure. It is however pointed out that the overall electronics can be arranged directly on the orthosis for the sake of simplicity. In such a case, an acoustically, vibrationally and/or optically active alarm can be generated on the orthosis and/or, in the event of an alarm, preferably wireless transmission to an alarm transmitter can be triggered, such as to a smartphone or a smartwatch.

By virtue of the invention, it is possible to guarantee a high level of safety with at the same time great comfort for the user in an energy-saving manner and with little effort.

What has been described above is thus, inter alia, but not exclusively, a loading-detecting orthosis having a standard sensor that generates sensor signals and that has no more than four pressure areas and no more than four sensor elements per pressure area, sensor signal-evaluating electronics therefor, and individual adaptation to individual patients, wherein the sensor signal-evaluating electronics are designed to indicate loading critical for the individual, wherein the sensor signal-evaluating electronics are designed to sample the pressure loading of the sensor elements at least per pressure area and to weight them, to run through a step identification, to classify load peaks for each step through comparison with a multiplicity of threshold values and to form a sum value into which load peaks are incorporated in weighted form such that higher load peaks receive higher weightings, and to generate an alert or warning signal when the sum value exceeds a particular amount, that is to say becomes too large.

1-10. (canceled)

11. A loading-detecting orthosis comprising:
a sensor; and
sensor signal-evaluating electronics;
wherein:
the sensor includes sensor elements in a plurality of pressure areas and configured to generate sensor signals; and
the sensor signal-evaluating electronics are configured to indicate critical loading by performing the following:
ascertaining characteristic values regarding pressure loading events detected, at least per pressure area of the plurality of pressure areas, by way of the sensor elements;
weighting the characteristic values using at least three different weights to form weighted characteristic values;
forming a sum value based on the weighted characteristic values; and
generating an alert signal when the sum value exceeds a stipulated amount.

12. The loading-detecting orthosis of claim 11, wherein the sensor signal-evaluating electronics are furthermore designed such that characteristic values are classified through comparison with a multiplicity of threshold values, and the weighting is performed based on the classification such that greater weightings are obtained as the characteristic values become larger.

13. The loading-detecting orthosis of claim 12, wherein the classification is performed to obtain a single classified characteristic value per user step.

14. The loading-detecting orthosis of claim 13, wherein the sensor signal-evaluating electronics are configured to perform the weighting based on a temporal density of respective ones of the characteristic values that are above one or more predefined threshold values.

15. The loading-detecting orthosis of claim 12, wherein the sensor signal-evaluating electronics are configured to perform the weighting based on a temporal density of respective ones of the characteristic values that are above one or more predefined threshold values.

16. The loading-detecting orthosis of claim 15, wherein the weighting based on the temporal density is performed such that contributions from pressure loading events to the sum value are caused to subside over time.

17. The loading-detecting orthosis of claim 16, wherein the weighting based on the temporal density is performed such that the smaller or shorter the pressure loading event, the faster the contribution by the respective pressure loading event subsides.

18. The loading-detecting orthosis of claim 12, wherein:
the sensor is part of an insole;
the plurality of pressure areas include no more than four pressure areas; and
the sensor signal-evaluating electronics are configured to perform the weighting based on a temporal density of respective ones of the characteristic values that are above one or more predefined threshold values.

19. The loading-detecting orthosis of claim 18, wherein:
the sensor signal-evaluating electronics include first circuits and second circuits;
the second circuits are connected to the sensor elements via a wireless interface; and
the first circuits are positioned closer to the sensor elements than the second circuits.

20. The loading-detecting orthosis of claim 19, wherein the wireless interface is a Bluetooth and/or WiFi interface.

21. The loading-detecting orthosis of claim 12, wherein the sensor signal-evaluating electronics are configured to combine values weighted by pressure area before the ascertaining of the characteristic values.

22. The loading-detecting orthosis of claim 11, wherein the sensor is standardized for a particular shoe size or shoe size group.

23. The loading-detecting orthosis of claim 22, wherein the sensor is part of an insole.

24. The loading-detecting orthosis of claim 11, wherein the plurality of pressure areas include no more than four pressure areas and no more than four sensor elements per pressure area.

25. The loading-detecting orthosis of claim 11, wherein the sensor signal-evaluating electronics are configured to obtain the characteristic values through A/D conversion of pressure loading sensor element signals and to weight them at least by pressure area.

26. An arrangement comprising:
an insole having a sensor for detecting a load; and
sensor signal-evaluating electronics;
wherein:
the sensor includes sensor elements in a plurality of pressure areas and configured to generate sensor signals; and
the sensor signal-evaluating electronics are configured to indicate critical loading by performing the following:
ascertaining characteristic values regarding pressure loading events detected, at least per pressure area of the plurality of pressure areas, by way of the sensor elements;
weighting the characteristic values using at least three different weights to form weighted characteristic values;
forming a sum value based on the weighted characteristic values; and
generating an alert signal when the sum value exceeds a stipulated amount.

27. The arrangement of claim 26, wherein the insole is adapted to equalize loading and/or damp pressure peaks.

28. The arrangement of claim 27, wherein:
the sensor signal-evaluating electronics include first circuits and second circuits;
the second circuits are connected to the sensor elements via a wireless interface; and
the first circuits are positioned closer to the sensor elements than the second circuits.

29. The arrangement of claim 26, wherein the sensor signal-evaluating electronics are configured to perform the weighting based on a temporal density of respective ones of the characteristic values that are above one or more predefined threshold values.

30. The arrangement of claim 29, wherein:
the sensor signal-evaluating electronics include first circuits and second circuits;
the second circuits are connected to the sensor elements via a wireless interface;
the first circuits are positioned closer to the sensor elements than the second circuits; and
the insole is adapted to equalize loading and/or damp pressure peaks.

31. The arrangement of claim 30, wherein the weighting based on the temporal density is performed such that contributions from pressure loading events to the sum value are caused to subside over time.
